(19) United States
(12) Patent Application Publication
(10) Pub. No.: US 2025/0017749 A1
(43) Pub. Date: Jan. 16, 2025

(54) VASECTOMY REVERSAL DEVICE

(71) Applicant: MALE FERTILITY AND PEYRONIES'S CLINIC PLLC, Orem, UT (US)

(72) Inventor: Landon Trost, Orem, UT (US)

(21) Appl. No.: 18/709,840

(22) PCT Filed: Nov. 4, 2022
(86) PCT No.: PCT/US2022/048949

§ 371 Date: May 14, 2024

Related U.S. Application Data
(60) Provisional application No. 63/281,651, filed on Nov. 20, 2021.

(51) Int. Cl. A61F 2/90; A61F 2/04

(52) U.S. Cl. CPC A61F 2/90; A61F 2/042

(57) ABSTRACT

Apparatuses, methods, and systems for vasectomy reversal are provided. An example vasectomy reversal stent includes body having a first end, a second end, and a channel extending between the first and second ends. The vasectomy reversal stent includes a flange extending radially outwardly from the body.

### TECHNICAL FIELD

A vasectomy is a procedure for male sterilization. It is commonly used as a form of birth control. Vasectomy involves surgically severing each vas deferens to prevent the passage

### BACKGROUND

A vasectomy is a procedure for male sterilization. It is commonly used as a form of birth control. Vasectomy involves surgically severing each vas deferens to prevent the passage of sperm from the testicles to the urethra. The vas deferens is a muscular tube that connects epididymis of a testicle to the urethra. After being severed the free ends of the vas deferens may be tied off or left free.

Vasectomy is a highly effective form of birth control and is usually permanent. A portion of patients who undergo a vasectomy procedure eventually end up wanting to reverse the procedure. Typically, the patient wants to reverse a vasectomy in order to restore fertility. In some situations, patients may want to reverse a vasectomy due to experiencing persistent pain after the vasectomy.

A vasectomy reversal is performed by either connecting the two ends of the severed vas to one another (vasovasostomy, or VV), or through connecting the vas deferens to the epididymis (epididymovasostomy, vasoepididymostomy, or EV or VE). The specific procedure performed is determined at the time of surgery and depends on intra-operative factors. This reconnection process may be challenging for many reasons. First, the vas deferens is quite small (e.g., typically 3-5 millimeters in diameter) and suturing these small structures is time consuming, high-precision work. Second, the reconnection may be more difficult because the diameters of the separated ends of the vas deferens may be mismatched due to sperm buildup dilating the end that remains attached to the epididymis. Third, even when successfully reconnected, scar tissue may reduce or completely block sperm passage through the reconnected vas deferens. Fourth, even when successfully reconnected, the repair may pull apart due to forces being applied to the repair with physical activities.

### SUMMARY

In general terms, this disclosure is directed to a device for vasectomy reversal. In one possible configuration and by non-limiting example, includes a vasectomy reversal stent for surgical implantation in a vas deferens. The vasectomy reversal stent may, for example, prevent scar tissue from blocking a rejoined vas deferens. In another non-limiting example, a stent is secured in the abdominal vas deferens only, which is then connected to either the epididymis or vas deferens. The system may also include an external brace (or support structure), which is sutured from the vas to the vas and/or epididymis to provide additional strength and reduce the likelihood of the repair pulling apart.

One aspect is a vasectomy reversal stent comprising an elongate tube having a first side and a second side that are separated by a transition region, wherein a maximum exterior diameter of the first side is greater than a maximum exterior diameter of the second side; and a loop coupled to an exterior surface of the elongate tube, the loop configured to receive a suture to couple the elongate tube to a vas deferens of a patient. Another aspect is a vasectomy reversal stent comprising an elongate structure having a first open end and a second open end, the elongate structure including a plurality of channels extending from the first open end to the second open end and configured to allow a fluid flow from the first open end to the second open end.

Yet another aspect is a vasectomy reversal stent comprising an elongate structure having a first open end and a second open end, the elongate structure including a plurality of channels extending from the first open end to the second open end and configured to allow a fluid flow from the first open end to the second open end; and a flange adjacent to the second open end, wherein the flange extends radially out from the elongate structure.

Another aspect is a vasectomy reversal stent comprising: an elongate tube having a first side and a second side; and a loop coupled to an exterior surface of the elongate tube, the loop configured to receive a suture to couple the elongate tube to a vas deferens of a patient.

Yet another aspect is a vasectomy reversal kit comprising: a first vasectomy reversal stent that includes an elongate structure having a first open end and a second open end, the first end and second end being tapered; and a second vasectomy reversal stent that includes an elongate structure having a first open end and a second open end, the first end and second end being tapered, wherein the length of the first vasectomy reversal stent is different than the length of the second vasectomy reversal stent.

The details of one or more aspects are set forth in the accompanying drawings and description below. Other features and advantages will be apparent from a reading of the following detailed description and a review of the associated drawings. It is to be understood that the following detailed description is explanatory only and is not restrictive of this disclosure.

### BRIEF DESCRIPTION OF THE DRAWINGS

FIG. 1 is a schematic diagram of an example vasectomy reversal device that is positioned to reconnect a vas deferens in accordance with teachings of the present disclosure.

FIG. 2 is a schematic diagram of an example vasectomy reversal device in accordance with embodiments described herein.

FIG. 3 is a schematic diagram of an example vasectomy reversal device in accordance with embodiments described herein.

FIG. 4A is a perspective view of an example vasectomy reversal device in accordance with embodiments described herein.

FIG. 4B is a cross-sectional view of the example vasectomy reversal device of FIG. 4A, taken at A-A.

FIG. 5 is a schematic diagram of an example vasectomy reversal device that is positioned to reconnect the vas deferens in accordance with embodiments described herein.

FIG. 6 is a schematic diagram of an example external support structure in accordance with embodiments described herein.

FIG. 7A is a schematic diagram of an example vasectomy reversal device that is positioned to maintain fluid flow when the vas deferens is directly reconnected to the epididymis of the testicle or to the convoluted/non-convoluted portions of the vas deferens in accordance with embodiments described herein.

FIG. 7B is a partial, magnified view of the example vasectomy reversal device of FIG. 7A, showing sutures connecting the device to the vas deferens.

FIG. 8A is a side, cross-sectional view of an example vasectomy reversal device in accordance with embodiments described herein.

FIG. 8B is a bottom view of the example vasectomy reversal device of FIG. 8A.

FIG. 9 is a schematic diagram of an example vasectomy reversal device that is positioned to reconnect a vas deferens.

FIG. 10 is a schematic diagram of another example vasectomy reversal device that is positioned to reconnect a vas deferens.

FIG. 11A is a perspective view of an example vasectomy reversal device in accordance with the teachings of the present disclosure.

FIG. 11B is a bottom view of the example vasectomy reversal device of FIG. 11A.

FIG. 11C is a side view of the example vasectomy reversal device of FIG. 11A.

FIG. 12A is a perspective view of an example vasectomy reversal device in accordance with the teachings of the present disclosure.

FIG. 12B is a bottom view of the example vasectomy reversal device of FIG. 12A.

FIG. 12C is a side view of the example vasectomy reversal device of FIG. 12A.

### DETAILED DESCRIPTION

Various embodiments will be described in detail with reference to the drawings, wherein like reference numerals represent like parts and assemblies throughout the several views. Reference to various embodiments does not limit the scope of this disclosure. Additionally, any examples set forth in this specification are not intended to be limiting and merely set forth some of the many possible embodiments for this disclosure.

The present disclosure relates to a stent for use in a vasectomy reversal procedure. Various subtypes of procedures to reverse a vasectomy are referred to as a vasovasostomy, epididymovasostomy, or vasoepididymostomy. As some of the embodiments may be used for either a vasovasostomy or epididymovasostomy/vasoepididymostomy, the stents used for vasectomy reversal procedures will be referred to herein as a vasectomy reversal stent.

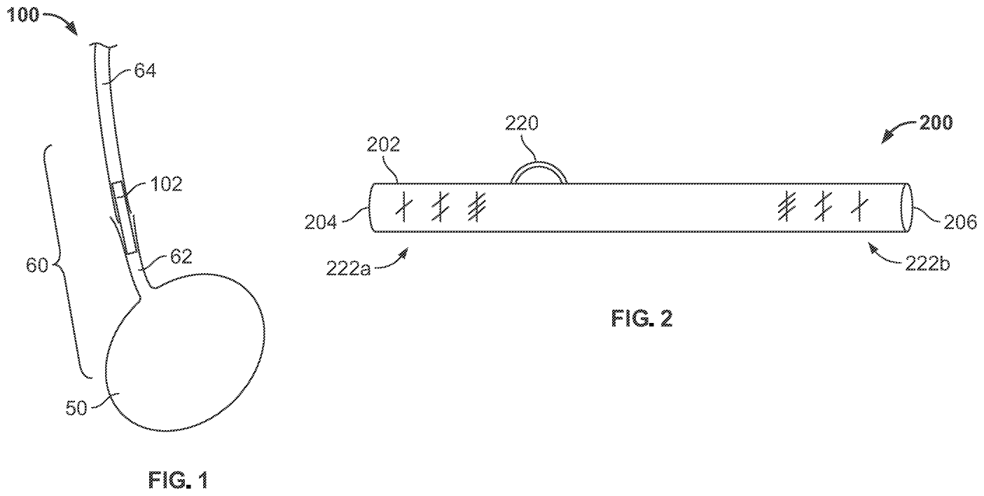

FIG. 1 is a schematic diagram of an example vasectomy reversal device 100 that is usable to reconnect a vas deferens 60. In this example, the device 100 includes a stent 102 that joins a first portion 62 of the vas deferens 60 to a second portion 64 of the vas deferens 60. The first portion 62 connects to the epididymis of a testicle 50. The second portion 64 extends into the abdomen and eventually to the urethra (not shown).

In this example, the vas deferens 60 had previously been severed. For example, the vas deferens may have been severed during a vasectomy procedure to prevent the passage of sperm produced in the testicle 50 to the urethra. Here, the device 100 is positioned to reconnect the first portion 62 to the second portion 64, restoring a passage for sperm produced in the testicle 50 to flow to the urethra. The stent 102 may be surgically placed during a vasectomy reversal procedure. Although not shown in this figure, the device 100 may include other components in addition to the stent 102 as will be described in more detail herein.

In this example, the stent 102 has an elongate tube shape that fluid, including sperm, may pass through. The elongate tube may have a first open end that is received within the first portion 62 and a second open end that is received within the second portion 64. The first end, the second end, or both may be tapered to allow for easier insertion within the open ends of the portions of the vas deferens 60.

In some implementations, the device 100 is secured to one or both of the first portion 62 and the second portion 64 with a suture. For example, the device 100 may include a loop structure (not shown) on the outer surface of the tube that a suture can pass through. The suture may secure the device 100 in place relative to the vas deferens 60 so as to prevent movement of the device 100 within the vas deferens.

Some implementations also include an external support structure (not shown) that may be placed on the exterior surface of the vas deferens, crossing the surgical anastomosis in a longitudinal direction. The external support structure may be secured to the vas deferens with one or more sutures. For example, the external support structure may include one or more apertures that a suture may pass through. Beneficially, the external support structure may provide additional support to the reconnected vas deferens, reducing the likelihood that that previously severed ends will pull apart from one another.

In some implementations of the device 100, the stent 102 has a uniform diameter. In some implementations, the stent 102 includes sides having different diameters to accommodate changes in the diameters of the portions of the vas deferens. The stent 102 may include a transition area between the sides that have different diameters.

In some implementations, the stent 102 includes multiple internal channels that fluid, including sperm, may flow through. For example, the stent 102 may include an elongate central hub that extends through the center (middle) of the elongate tube. The elongate central hub may be joined to the perimeter of the hub with fins that also extend out from the central hub to the perimeter. Multiple fins may be evenly spaced around the central hub. For example, four fins may be spaced apart from each other by approximately 90 degrees and may extend out from the central hub to the perimeter of the elongate tube. In some implementations, the elongate tube may include openings disposed between the fins that extend longitudinally at least part way from the first open end to the second open end. The openings may extend fully from the first open end of the device 100 to the second open end.

In some implementations, the stent 102 may include a drug-eluting layer or coating that releases a drug or chemical compound to reduce the likelihood of tissue forming around or within the stent 102 and potentially occluding passage through the stent 102. At least a portion of the stent 102 may be covered with a polymer coating that binds and releases a drug. For example, the drug may be an immunosuppressive or antiproliferative drug. Non-limiting examples of drugs that may be used in some embodiments include paclitaxel, zotarolimus, everolimus, and sirolimus.

FIG. 2 is a schematic diagram of an example vasectomy reversal device 200. The vasectomy reversal device 200 is an example of the device 100. In this example, the device 200 includes a stent 202 and a loop 220 coupled to the exterior surface of the stent 202.

The stent 202 is a hollow tubular structure that extends longitudinally from a first open end 204 to a second open end 206. The stent 202 is sized to fit within a vas deferens. For example, the stent 202 may have an outer diameter of between 0.6 millimeters and 1.6 millimeters. In some implementations, the outer diameter of the stent 202 is approximately 0.7 millimeters. The walls of the stent 202 may have a thickness of approximately 0.1-0.3 millimeters.

In this example, the loop 220 is coupled to the exterior surface of the stent 202. The loop may, for example, be made of the same material as the stent 202. The loop 220 in combination with the exterior surface of the stent 202 forms an opening that a suture can be passed through to secure the device 200 at a specific location in the vas deferens. The suture passing through the loop 220 may secure the device 200 at the location in the vas deferens has been repaired so that the stent is better situated to prevent scar tissue from building up to block the passage of fluid. For example, the suture passing through the loop 220 may prevent the device 200 from sliding (moving) through the inside of the vas deferens.

In this example, the loop 220 is shown as being oriented in a direction that is parallel to the long axis of the stent 202 (i.e., the direction in which fluid flows through the stent 202). Alternatives are possible. For example, the loop 220 may be oriented perpendicular to the long axis of the stent 202, which may allow a surgeon use one suture to connect the two vasa and pass directly through the loop 220. The loop 220 may also be oriented at a diagonal with respect to the long axis of the stent 202.

In some implementations, the loop 220 is formed directly in the exterior surface of the stent 202. For example, the loop 220 is formed between two openings on the exterior surface of the stent 202, such as two parallel slits.

The stent 202 may be sized to fit within the interior channel (or duct) of a vas deferens. For example, the stent 202 may have an exterior diameter that is approximately equal to an interior diameter of a vas deferens and the length of the stent 202 may be less than a length of a generally straight (or non-convoluted) region of a vas deferens.

In some implementations, the stent 202 may have a length of between 2 to 14 millimeters. Although not shown in this figure, the stent 202 may have tapered or dilated ends. For example, the outer diameter of the stent 202 may gradually reduce or expand at the first open end 204 and the second open end 206. In some implementations, a longitudinally middle portion of the stent has a uniform diameter, while an end portion is tapered. The end portion may be, for example, 0.1-1 millimeter in length. The tapered ends may make it easier for the stent 202 to be inserted into the vas deferens.

In some implementations, the stent 202 is provided in a kit that includes multiple stents of differing lengths. As a non-limiting example, the kit may include a 2 millimeter stent, 5 millimeter stent, 8 millimeter stent, 10 millimeter stent, and a 12 millimeter stent. During a vasectomy reversal procedure, a surgeon may select an appropriate length stent from the kit. Beneficially, by providing a kit with stents having multiple lengths, the stent will not need to be cut to a size to fit the patient, which would remove at least one of the tapered ends. In another implementation, the stent may come in a larger length and may be cut to size by the surgeon at the time of the procedure. In this iteration, the length may be individually customized based on the surgeon's discretion and intra-operative factors. In some embodiments, the kit may include other components or other types of stents, such as external support structure of varying sizes and stents having multiple channels, flanges, or sides with different exterior diameters.

In this example, the stent 202 include an indictor region 222a located at one end and an indicator region 222b located at the other end. Collectively, the indicator region 222a and 222 may be referred to as the indicator regions 222. The indicator regions 222 include indicators of insertion depth. For example, the indicator regions 222 may include multiple visual indicators disposed at different distances from the corresponding end of the stent. During a surgical procedure, the surgeon may be able to look at one of the indicator regions 222 to quickly estimate how much of the stent 202 has been inserted into the vas deferens. In this example, the indicator regions 222 each include three equally spaced visual indicators. The visual indicators have different numbers of hash marks so that the surgeon only needs to see one of the visual indicators to estimate distance to the end of the stent 202. For example, the visual indicator with 1 hash mark may be 1 millimeter from the end of the stent, the visual indicator with 2 hash marks may be 2 millimeters from the end of the stent, and the visual indicator with 3 hash marks may be 2 millimeters from the end of the stent. Some implementations include more, fewer, or different types of visual indicators. The visual indicators may be formed by etching markings into the exterior surface of the stent 202, applying ink to the exterior surface of the stent 202, forming ridges on the exterior surface of the stent 202, or embedding a visually distinctive material within or on the surface of the stent 202.

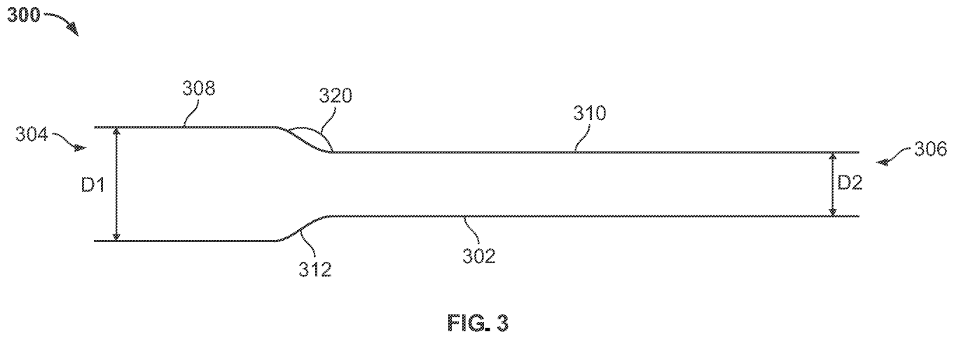

FIG. 3 is a schematic diagram of an example vasectomy reversal device 300. The vasectomy reversal device 300 is an example of the device 100. In this example, the device 300 includes a stent 302 and a loop 320 coupled to the exterior surface of the stent 302. The stent 302 may be similar to the stent 202 that has been previously illustrated and described with respect to at least FIG. 2 except that the stent 302 does not have a uniform diameter.

The stent 302 is a hollow tubular structure that extends longitudinally from a first open end 304 to a second open end 306. Here, the stent 302 includes a first portion 308, a second portion 310, and a transition portion 312. The first portion 308 extends from the first open end 304 to the transition portion 312. The second portion 310 extends from the second open end 306 to the transition portion 312. The first portion 308 and the second portion 310 do not have the same diameter. The transition portion 312 may have a variable diameter that transitions from the greater diameter of the first portion 308 the lesser diameter of the second portion 310. In some implementations, the diameter of the transition portion 312 varies linearly from the diameter of the first portion 308 to the second portion 310.

In this example, diameter of the first portion 308 is greater than the diameter of the second portion 310. The diameter of the first portion 308 may be 150%, 200%, 300% or more of the diameter of the second portion 310. In some implementations, the length of the first portion 308 is equal to or approximately equal to the length of the second portion 310. In some implementations, the length of the first portion 308 (i.e., the longitudinal distance from the first end 304 to the interface between the first portion 308 and the transition portion 312) is less than the length of the second portion 310 (i.e., the longitudinal distance from the second end 306 to the interface between the second portion 310 and the transition portion 312). For example, the length of first portion 308 may be 75%, 50%, 33%, 25%, 20%, or less of the length of the second portion 310. Although alternatives are possible, in some implementations, the length of the first portion 308 is 0.25 centimeters and the length of the second portion 310 is 1.0 centimeters.

The loop 320 may be similar to the previously described loop 220. In this example, the loop 320 is coupled to the exterior surface of the stent 302, extending from the transition portion 312 to the second portion 310. Beneficially, this positioning of the loop will be adjacent to the two ends of the severed vas deferens when the stent is placed. One or more sutures may be placed through the loop 320 to secure the stent 302 in place within the vas deferens. In some implementations, four sutures are used to rejoin end-to-end a first severed portion of the vas deferens to a second severed portion of the vas deferens and at least one of those sutures also passes through the loop 320.

Although not shown in this figure, one or both of the first portion 308 and the second portion 310 may include tapered ends, in which the diameter of the first portion 308 or second portion 310 tapers to reduced diameters at the first end 304 and the second end 306, respectively.

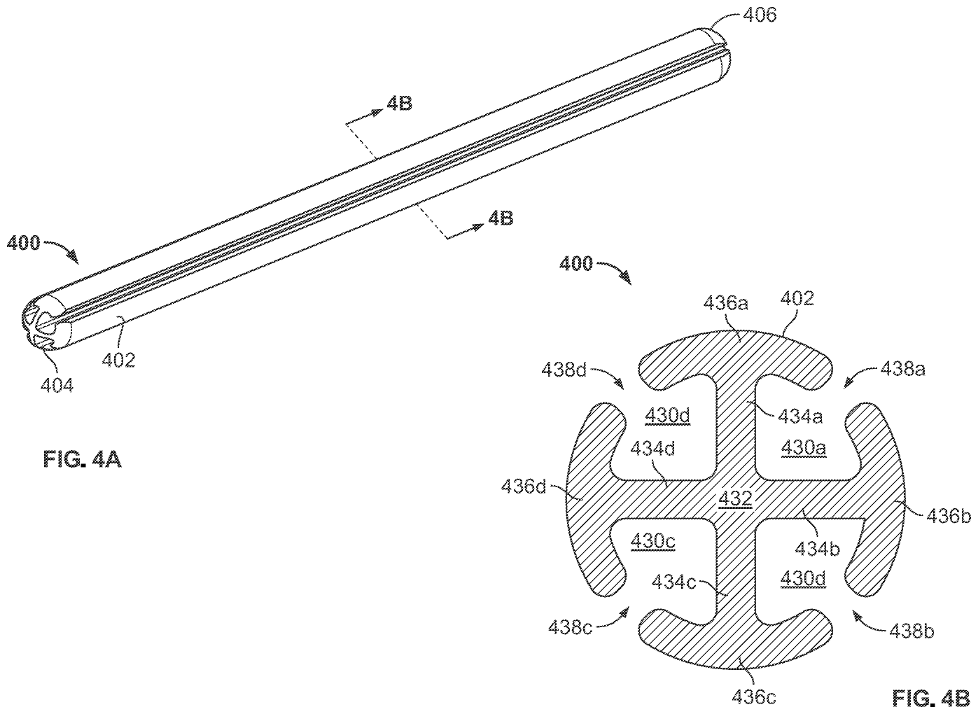

FIGS. 4A and 4B are schematic diagrams of an example vasectomy reversal device 400. FIG. 4A is a perspective view of the device 400 and FIG. 4B is a cross-sectional view of the device 400. The vasectomy reversal device 400 is an example of the device 100.

In this example, the device 400 includes a stent 402. The stent 402 has an approximately circular cross-sectional shape. Here, the stent 402 includes multiple channels that extend longitudinally from a first end 404 to a second end 406.

Beneficially, the multiple channels may provide additional routes for fluid passage even if one or more of the channels is blocked by scar tissue. These elongate openings may open into one or more of the channels.

In this example, the stent 402 includes channel 430a, 430b, 430c, and 430d (collectively, channels 430). The channels 430 are defined by an elongate central hub 432 with fin 434a, fin 434b, fin 434c, and fin 434d (collectively, fins 434) extending out from the central hub 432. Here, the stent 402 also includes wing structure 436a, 436b, 436c, and 436d (collectively, wing structures 436). Each of the wing structure 436 may be joined to the corresponding fins 434 at an end of fin 434 opposite the elongate central hub 432. Specifically, in this example, the wing structure 436a is joined to the fin 434a, the wing structure 436b is joined to the fin 434b, the wing structure 436c is joined to the fin 434c, and the wing structure 436d is joined to the fin 434d.

The wing structures 436 may have an arched shape that follows at least a portion of a circular perimeter of the stent 402. The wing structures 436 may extend out from the fins 434 at approximately right angles along the perimeter of the stent 402 towards an adjacent fin of the fins 434.

In some implementations, the perimeter of the stent 402 includes one or more elongate openings along its perimeter. These openings may be formed between pairs of adjacent wing structures from the wing structures 436. These opening may provide access to the channels 430.

For example, the stent 402 includes an opening 438a between the wing structure 436a and the wing structure 436b, an opening 438b between the wing structure 436b and the wing structure 436c, an opening 438c between the wing structure 436c and the wing structure 436d, and an opening 438d between the wing structure 436d and the wing structure 436a. In some implementations, each wing structure may each direction by extend approximately one third of the arc length distance toward. In this case, the opening will also occupy approximately one third of the arc length.

In some implementations, a suture may be passed through one or more of three openings 438 during surgery to secure the stent 402 within the reconnected vas deferens. For example, a suture may pass through one of the openings, through an adjacent fin, and back out through another opening. As a more specific example, the suture may pass through the opening 438a and into the channel 430a, through the fin 434 and into the channel 430d, and back out through the opening 438d.

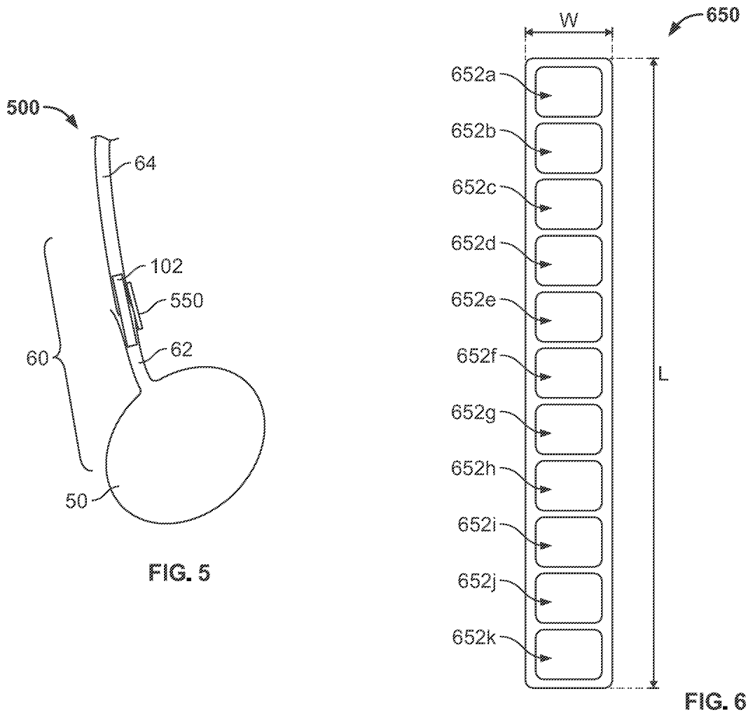

FIG. 5 is a schematic diagram of an example vasectomy reversal device 500 that is positioned to reconnect the vas deferens 60. In this example, the device 500 includes the stent 102 and an external support structure 550. The stent 102 has been previously described with respect to at least FIG. 1.

The external support structure 550 is a structure that is secured to the outside of the vas deferens after the vas deferens has been reconnected. The external support structure 550 may be secured to the vas deferens at multiple points with one or more sutures. For example, the external support structure 550 may be coupled to at least one point on the first portion 62 of the vas deferens 60 and at least one point on the second portion 64 of the vas deferens 60. In this manner, the external support structure 550 provides additional support to retain the vas deferens 60 in a reconnected state and to prevent (or reduce the likelihood) that the portions of the vas deferens 60 will reseparate. In some implementations, the external support structure 550 is coupled at multiple points to first portion 62 and at multiple points to the second portion 64.

In some implementations, the external support structure 550 includes one or more apertures that a suture may pass through. The external support structure 550 may formed from a thin piece of material that has a rectangular footprint. The apertures within the external support structure 550 may be arranged in a ladder or mesh-like structure.

The external support structure 550 may be made of one or more biocompatible materials that are suitable for being surgically implanted around the vas deferens. For example, the external support structure 550 may be made of a plastic or silicone material. In some implementations, the external support structure 550 is made from one or more of implantable grade silicone, polycarbonate-based thermoplastic polyurethanes such as CARBOTHANE™ or PELLETHANE™, thermoplastic elastomers such as PEBAX™, polyimide, polyetheretherketone (PEEK), polytetrafluoroethylene (PTFE), which may be marketed with the brand name TEFLON™, liquid crystal polymer (LCP), polypropylene, polyester, or nylon materials. The external support structure 550 may be designed to withstand at least 10 pounds of tensile pull without tearing. The external support structure 550 may be formed from a material that is substantially rigid. The external support structure 550 may be more rigid than the stent 102 in at least some implementations.

FIG. 6 is a schematic diagram of an example external support structure 650. The external support structure 650 is an example of the external support structure 550. In this example, the external support structure 650 has a rectangular footprint having a length L and width W. In some implementations, the support structure 650 has a length of between 10 and 50 millimeters. In some implementations, the support structure 650 has a length of between 20 and 45 millimeters. In some implementations, the support structure 650 has a length of between 30 and 40 millimeters. In some implementations, the support structure 650 has a length of between 35 and 40 millimeters. In some implementations, the support structure 650 has a length of between 38 and 40 millimeters. In some implementations, the support structure 650 has a length of approximately 39 millimeters. The support structure 650 may have a width of between 2 and 8 millimeters. In some implementations, the support structure 650 may have a width of between 3 and 6 millimeters. In some implementations, the support structure 650 may have a width of approximately 3 millimeters, approximately 4 millimeters, or approximately 5 millimeters.

The external support structure 650 may be formed from a thin piece of rigid material. For example, the support structure 650 may have a thickness of between 0.01 millimeters and 2 millimeters. The support structure 650 may have a thickness of between 0.2 millimeters and 1 millimeter. Although alternatives are possible, the support structure 650 may have a thickness of approximately 0.5 millimeters.

In this example, the external support structure 650 includes an aperture 652a, aperture 652b, aperture 652c, aperture 652d, aperture 652e, aperture 652f, aperture 652g, aperture 652h, aperture 652i, aperture 652j, and aperture 652k (collectively, apertures 652). Other embodiments are possible that include more or fewer apertures. In this example, the apertures may be sized so that at least one suture may be passed through during surgery to implant the external support structure 650. In some implementations, sutures may be passed through a first plurality of the apertures 652 to secure the external support structure 650 to a first portion of the vas deferens and through a second plurality of the apertures 652 to secure the external support structure 650 to a second portion of the vas deferens. In this manner, the external support structure 650 is coupled to both portions of the reconnected vas deferens, providing support and resistance against pulling forces that may otherwise cause the portions of the vas deferens to separate over time.

For example, sutures may pass through the apertures 652a, 652b, 652c, 652d, and 652e to secure the external support structure 650 to a first portion of the vas deferens, and sutures may pass through the apertures 652g, 652h, 652i, 652j, and 652k to secure the external support structure 650 to a second portion of the vas deferens. Of course, other numbers of sutures and apertures may be used to couple the external support structure 650 to portions on the vas deferens. In at least some embodiments, not all of the apertures 652 are used to secure external support structure 650 to the vas deferens.

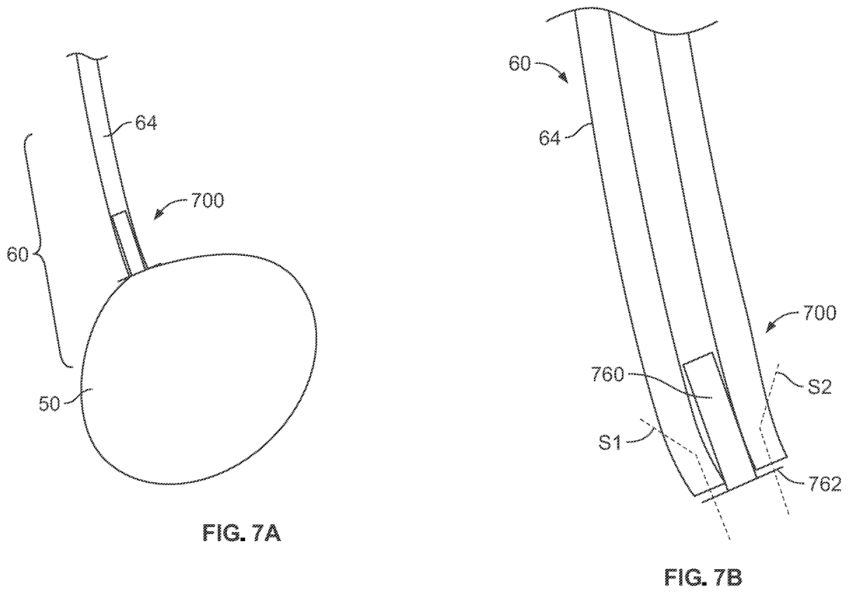

FIGS. 7A and 7B are schematic diagrams of an example vasectomy reversal device 700 that is positioned to maintain fluid flow when the vas deferens 60 is directly reconnected to the epididymis of the testicle 50. The vas deferens 60 may be connected directly to the epididymis of the testicle 50 because, for example, an obstruction may be present in another region along the vas deferens or portion of the epididymis which would then require reconnecting to a higher up portion of the epididymis.

FIG. 7A shows a view of the second portion 64 of the vas deferens 60 connected to the epididymis of the testicle 50 with the vasectomy reversal device 700 surgically positioned within the second portion 64. The vasectomy reversal device 700 is positioned at the end of the second portion 64, adjacent to an interface with the epididymis of the testicle 50.

FIG. 7B is a close-up view of the second portion 64 of the vas deferens 60 and the vasectomy reversal device 700. In this example, the vasectomy reversal device 700 includes a stent portion 760 and a flange portion 762. The stent portion 760 may be similar to the previously described stent 102. The flange portion 762 may project outward at approximately a perpendicular angle from and end of the stent portion 760. The flange portion 762 may be used to surgically secure the vasectomy reversal device 700 to an end of the second portion 64 of the vas deferens 60.

In this example, sutures S1 and S2 are shown passing through the flange portion 762 and into and through the second portion 64 of the vas deferens 60. Here, the sutures S1 and S2 pass through the face of the vas deferens 60 (i.e., the surface of the vas deferens 60 adjacent to the flange portion 762) and out the side (i.e., the curved outer surface of the vas deferens 60). In this manner, the flange portion 762 secures the device 700 to the end of the second portion 64 of the vas deferens 60, positioning the stent portion 760 where it may prevent scarring and closure (or occlusion) of the second portion 64 of the vas deferens 60 so that fluid, including sperm, may flow from the testicle 50 into and through the vas deferens 60. In some implementations, the flange portion 762 may be attached to the vas deferens with sutures that pass into and out of the face of the vas deferens 60.

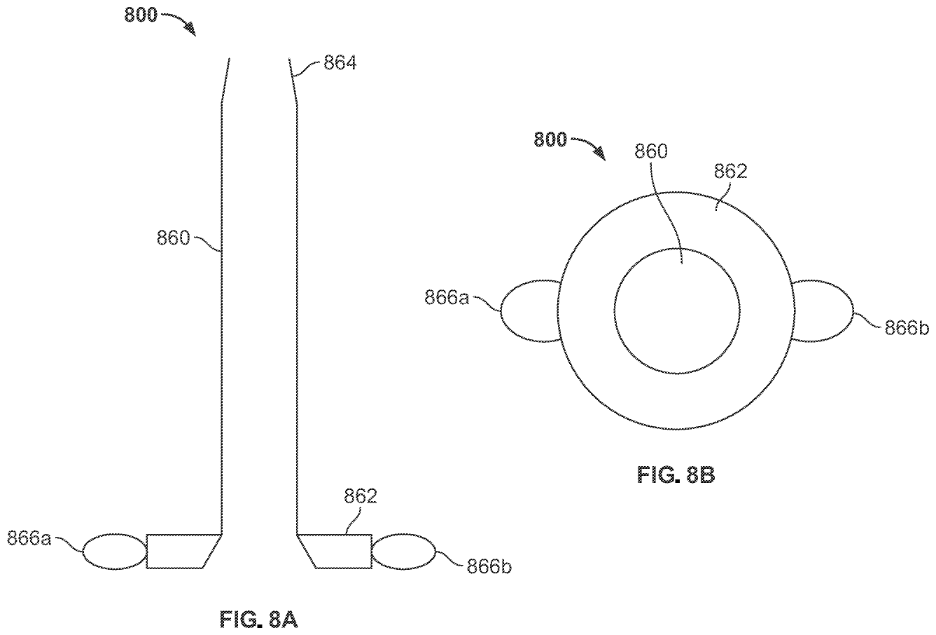

FIGS. 8A and 8B are schematic diagrams of an example vasectomy reversal device 800. The device 800 may be similar to the device 700, which was previously described with respect to at least FIGS. 7A and 7B.

The device 800 includes a stent 860 and a flange 862. One end of the stent 860 is joined to the flange 862. The stent 860 may also include a straight or tapered end 864 that is opposite the flange 862. In the case of a tapered end 864, it may exhibit a gradually reduced diameter to facilitate easier insertion of the stent 860 into the open end of the second portion 64 of the vas deferens.

The flange 862 is a structure that extends out radially from one end of the exterior surface of the stent 860. The flange 862 may be a thin structure. For example, the flange may be between 0.05 millimeter and 2 millimeters thick. In some implementations, the radial length of the flange (i.e., distance the flange extends from the interface with the stent 860 to its outer edge) may cover only a portion of the wall thickness. For example, the radial length may be 0.1-0.5 millimeters. In some implementations, the radial length of the flange may be approximately equal to half of the thickness of the walls of vas deferens. For example, the radial length of the flange 862 may be between 0.5 millimeters and 2 millimeters. Beneficially, if the flange 862 does not extend fully to the outer edge of the wall of the vas deferens, a portion of the face of the vas deferens will be in contact with the epididymis, facilitating the vas deferens scarring into the epididymis.

The flange 862 may also include one or more loops. Here, the flange includes a loop 866a and a loop 866b (collectively loops 866) extending out from the outer perimeter. Some implementations may include more than two of the loops 866. The loops 866 include openings that a suture may pass through to secure the device 800 to the vas deferens. In this manner, the stent 860 of the device 800 is held in place by the sutures that pass through the loops 866 so that the stent 860 can prevent closure (or occlusion) of the vas deferens caused by, for example, scar tissue formation or tissue ingrowth.

In some implementations, the flange 862 may include one or more apertures that sutures may be passed through to secure the device 800 to the vas deferens 60. For example, a suture may be passed through one of the apertures and into the vas deferens.

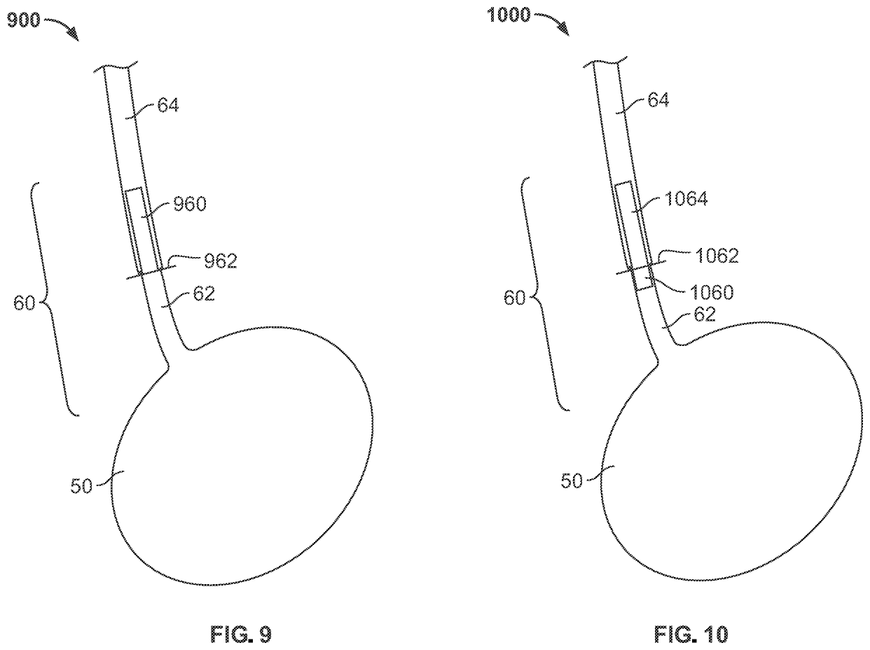

FIG. 9 is a schematic diagram of an example vasectomy reversal device 900 that is positioned to reconnect a vas deferens 60. In this example, the device 900 joins a first portion 62 of the vas deferens 60 to a second portion 64 of the vas deferens 60. The first portion 62 connects to the epididymis of a testicle 50. The second portion 64 extends into the abdomen and eventually to the urethra (not shown). The vasectomy reversal device 900 may be similar to the vasectomy reversal device 700 that is illustrated and described with reference to at least FIGS. 7A and 7B.

The vasectomy reversal device 900 includes a stent portion 960 and a flange portion 962. The stent portion 960 may be similar to the previously described stent portion 760 and the flange portion 962 may be similar to the previously described flange portion 762. The flange portion 962 may project outward at approximately a perpendicular angle from an end of the stent portion 960.

The flange portion 962 may be used to surgically secure the vasectomy reversal device 900 to an end of the second portion 64 of the vas deferens 60. Sutures may pass through the flange portion 962 and into and through the second portion 64 of the vas deferens 60. In this manner, the flange portion 962 secures the device 900 to the end of the second portion 64 of the vas deferens 60, positioning the stent portion 960 where it may prevent scarring and closure (or occlusion) of the second portion 64 of the vas deferens 60 so that fluid, including sperm, may flow from the testicle 50 into and through the vas deferens 60. Sutures may then also be used to connect the first portion 62 of the vas deferens 60 to the flange portion 962 or to the second portion 64 of the vas deferens.

It should be understood that the flange portion 962 may extend out from the exterior surface of the stent portion 960 by different amounts in different embodiments. Although the flange portion 962 may appear to extend out beyond the exterior diameter of the vas deferens 60 in this figure, it should be understood that in at least some embodiments, the flange portion 962 may not extend past the outer diameter of the vas deferens 60. For example, the flange portion 962 may extend out to approximately half the thickness of the vas deferens 60 so that the vas portions may be in contact and heal (scar) together. In an example, the inner diameter of the vas deferens 60 may be approximately 0.5 mm, and the outer diameter of the lumen of the vas deferens 60 may be approximately 4 mm, the flange portion 962 may extend out by 1 mm on all sides. The thickness of the walls of the vas deferens 60 is not shown in this figure so that the flange portion 962 is more clearly illustrated.

FIG. 10 is a schematic diagram of an example vasectomy reversal device 1000 that is positioned to reconnect a vas deferens 60. In this example, the device 1000 joins a first portion 62 of the vas deferens 60 to a second portion 64 of the vas deferens 60. The first portion 62 connects to the epididymis of a testicle 50. The second portion 64 extends into the abdomen and eventually to the urethra (not shown). The vasectomy reversal device 1000 may be similar to the vasectomy reversal device 900 that is illustrated and described with reference to at least FIG. 9.

Here, the vasectomy reversal device 1000 includes a first stent portion 1060, a second stent portion 1064, and a flange portion 1062 disposed between the first stent portion 1060 and the second stent portion 1064. The second stent portion 1064 may be similar to the previously described stent portion 960 and the flange portion 1062 may be similar to the previously described flange portion 962. The first stent portion 1060 may be similar to the second stent portion 1064 except that it is disposed across the flange portion 1062 from the second stent portion 1064. In some implementations, the first stent portion 1060 may have a shorter length than the second stent portion 1064. For example, the length of the first stent portion 1060 may be less than 50% of the length of the second stent portion 1064. In some implementations, the length of the first stent portion 1060 is equal to or approximately equal to 25% of the length of the second stent portion 1064.

For example, the length of the first stent portion 1060 may be equal to 20-30% of the length of the second stent portion 1064. The flange portion 1062 may project outward at approximately a perpendicular angle from exterior surface of the first stent portion 1060 or the second stent portion 1064.

The flange portion 1062 may be used to surgically secure the vasectomy reversal device 1000 to an end of the second portion 64 of the vas deferens 60 or the first portion 62 of the vas deferens 60. Sutures may pass through the flange portion 1062 and into and through the second portion 64 of the vas deferens 60 or the first portion 62 of the vas deferens 60. In this manner, the flange portion 1062 secures the device 1000 to the end of the second portion 64 of the vas deferens 60, positioning the first stent portion 1060 where it may prevent scarring and closure (or occlusion) of the first portion 62 of the vas deferens 60 and the second stent portion 1064 where it may prevent scarring and closure (or occlusion) of the second portion 64 of the vas deferens 60, so that fluid, including sperm, may flow from the testicle 50 into and through the vas deferens 60. Sutures may then also be used to connect the first portion 62 of the vas deferens 60 to the flange portion 1062 or to the second portion 64 of the vas deferens.

An elongate tube is an example of a stent portion. A first side of an elongate tube is an example of a first stent portion and a second side of an elongate tube may be an example of a second stent portion.

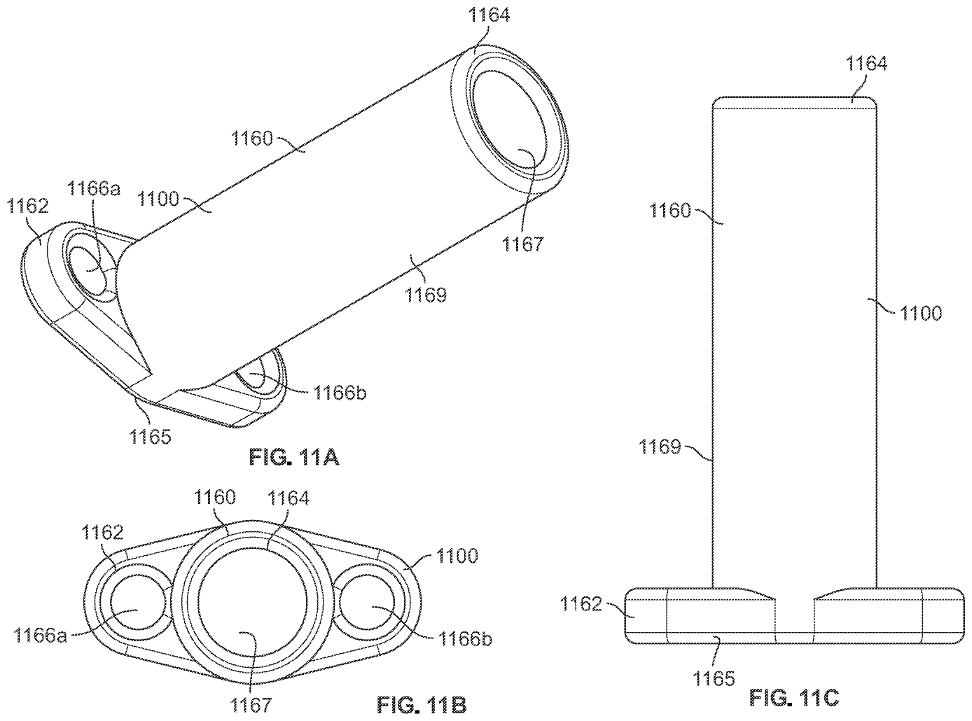

FIGS. 11A-11C are schematic diagrams of an example vasectomy reversal device 1100. The device 1100 may be similar to the device 800, which was previously described with respect to at least FIGS. 8A and 8B.

The device 1100 includes a stent or body 1160 and a flange 1162 extending radially outwardly from the body 1160. The body 1160 includes a first end 1164, a second opposite end 1165. The first and second ends 1164, 1165 may be in fluid communication by way of a channel 1167 extending between the first and second ends 1164, 1165. The second end 1165 of the stent 1160 is joined to the flange 1162. The stent 1160 may also include a straight or tapered end 1164 that is opposite the flange 1162. In the case of a tapered end 1164, it may exhibit a gradually reduced diameter to facilitate easier insertion of the stent 1160 into the open end of the second portion 64 of the vas deferens 60.

The flange 1162 is a structure that extends out radially from the second end 1165 of an exterior surface 1169 of the stent 1160. The flange 1162 may be a thin structure. For example, the flange may be between 0.005 millimeters and 0.1 millimeters thick. In some implementations, the radial length of the flange (i.e., distance the flange extends from the interface with the stent 1160 to its outer edge) may cover only a portion of the wall thickness. In some implementations, the radial length of the flange may be approximately equal to half of the thickness of the walls of vas deferens. Beneficially, if the flange 1162 does not extend fully to the outer edge of the wall of the vas deferens, a portion of the face of the vas deferens will be in contact with the epididymis, facilitating the vas deferens scarring into the epididymis.

The flange 1162 may also include one or more apertures 1166. Here, the flange 1162 includes a first aperture 1166a and a second aperture 1166b (collectively apertures 1166). Some implementations may include more than two of the apertures 1166. The apertures 1166 define openings that a suture may pass through to secure the device 1100 to the vas deferens. In this manner, the stent 1160 of the device 1100 is held in place by the sutures that pass through the apertures 1166 so that the stent 1160 can prevent closure (or occlusion) of the vas deferens caused by, for example, scar tissue formation or tissue ingrowth. Each aperture 1166 may have a diameter in a range of approximately 0.005 mm to approximately 0.015 mm.

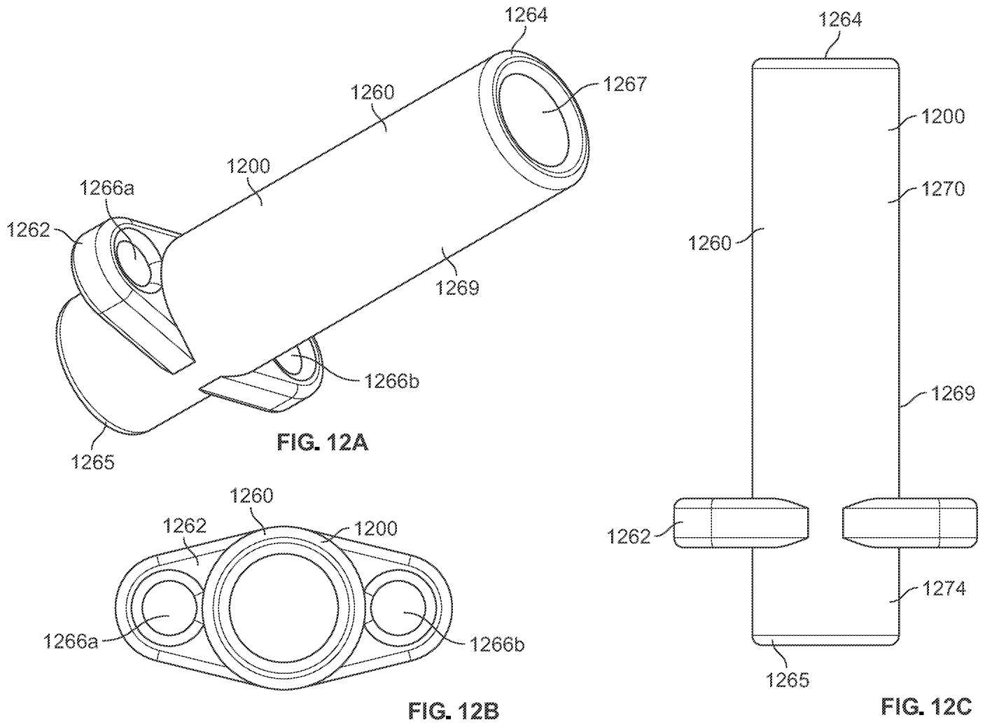

FIGS. 12A-12C are schematic diagrams of an example vasectomy reversal device 1200. The device 1200 may be similar to the device 1100, which was previously described with respect to at least FIGS. 11A-11C.

The stent 1260 includes a first portion 1270 defining a first open end 1264, and a portion 1274 end defining a second open end 1265. A flange 1262 extends radially outwardly from an exterior surface 1269 of the body 1260. The first portion 1270 extends away from the flange 1262 towards the first end 1264, and the second portion 1270 extends away from the flange 1262 in an opposite direction towards the second end. In some examples, a length defined between the flange 1262 and the first end may be in a range of approximately 0.03 mm to approximately 0.2 mm. In some examples, a length defined between the flange and the second end may be in a range of approximately 0.015 mm to approximately 0.06 mm. The stent 1260 may also include a straight or tapered end 1264, 1265. In the case of a tapered end, it may exhibit a gradually reduced diameter to facilitate easier insertion of the stent 1260 into the open end of the second portion 64 of the vas deferens 60.

The flange 1262 is a structure that extends out radially from the exterior surface 1269 of the body 1260. The flange 1262 may be a thin structure. For example, the flange may be between 0.005 millimeters and 0.1 millimeters thick. In some implementations, the radial length of the flange (i.e., distance the flange extends from the interface with the stent 1260 to its outer edge) may cover only a portion of the wall thickness. In some implementations, the radial length of the flange may be approximately equal to half of the thickness of the walls of vas deferens. Beneficially, if the flange 1262 does not extend fully to the outer edge of the wall of the vas deferens, a portion of the face of the vas deferens will be in contact with the epididymis, facilitating the vas deferens scarring into the epididymis.

The flange 1262 may also include one or more apertures 1266. Here, the flange 1262 includes a first aperture 1266a and a second aperture 1266b (collectively apertures 1266). Some implementations may include more than two of the apertures 1166. The apertures 1266 define openings that a suture may pass through to secure the device 1200 to the vas deferens. In this manner, the stent 1260 of the device 1200 is held in place by the sutures that pass through the apertures 1266 so that the stent 1260 can prevent closure (or occlusion) of the vas deferens caused by, for example, scar tissue formation or tissue ingrowth. Each aperture 1266 may have a diameter in a range of approximately 0.005 mm to approximately 0.015 mm.

The devices disclosed herein may be made of one or more biocompatible materials that are suitable for being surgically implanted in the vas deferens. For example, the device may be made of a plastic, silicone, or metallic material. In some implementations, the device is made from one or more of implantable grade silicone, polycarbonate-based thermoplastic polyurethanes such as CARBOTHANE™ or PELLETHANE™, thermoplastic elastomers such as PEBAX™, polyimide, polyetheretherketone (PEEK), polytetrafluoroethylene (PTFE), which may be marketed with the brand name TEFLON™, liquid crystal polymer (LCP), polypropylene, polyester, or nylon materials. In some examples, the device may be a titanium. In at least some implementations, the stent is formed from a material that human tissue does not usually grow on, such as silicone. Beneficially, in these embodiments, the stent may be less likely to blocked by scar tissue, vasal mucosal tissue, or other types of tissue which may grows in, through, or around it.

While this specification contains many specific implementation details, these should not be construed as limitations on the scope of any invention or of what may be claimed, but rather as descriptions of features that may be specific to particular examples of particular inventions. Certain features that are described in this specification in the context of separate examples can also be implemented in combination in a single example. Conversely, various features that are described in the context of a single example can also be implemented in multiple examples separately or in any suitable subcombination. Moreover, although features may be described herein as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the examples described herein should not be understood as requiring such separation in all examples, and it should be understood that the described program components and systems can generally be integrated together in a single product or packaged into multiple products.

Particular examples of the subject matter have been described. Other examples are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous.

1. A vasectomy reversal stent comprising:
a body having a first end, a second end, and a channel extending between the first and second ends; and
a flange extending radially outwardly from the body.

2. The vasectomy reversal stent of claim 1, comprising an aperture for receiving a suture.

3. The vasectomy reversal stent of claim 2, wherein the aperture is disposed through a portion of the flange.

4. The vasectomy reversal stent of claim 1, wherein the flange is disposed between a first portion and a second portion of the body.

5. The vasectomy reversal stent of claim 4, wherein a length of the first portion is less than a length of the second portion.

6. The vasectomy reversal stent of claim 4, wherein the length of the first portion is in a range between 20% to 30% of the length of the second portion.

7. The vasectomy reversal stent of claim 1, wherein the flange extends perpendicularly from an exterior surface of the body.

8. The vasectomy reversal stent of claim 1, comprising a loop coupled to the flange, the loop being sized to receive a suture to couple the body to a vas deferens of a patient.

9. The vasectomy reversal stent of claim 1, comprising a loop coupled to an exterior surface of the body, the loop configured to receive a suture to couple the body to a vas deferens of a patient.

10. The vasectomy reversal stent of claim 1, wherein the second end is tapered.

11. A vasectomy reversal stent comprising:
a tube having an exterior surface, a first portion having an exterior diameter, and a second portion having an exterior diameter;
a loop coupled to the exterior surface of the tube, the loop is configured to receive a suture to couple the tube to a vas deferens of a patient;
wherein the exterior diameter of the first portion is greater than the exterior diameter of the second portion.

12. The vasectomy reversal stent of claim 11, wherein the exterior diameter of the first portion is in a range of approximately 0.9 millimeters to approximately 3 millimeters.

13. The vasectomy reversal stent of claim 11, wherein the exterior diameter of the second portion is in a range of approximately 0.6 millimeters and approximately 0.8 millimeters.

14. The vasectomy reversal stent of claim 11, wherein the exterior diameter of the first portion is at least approximately 150% of the exterior diameter of the second portion.

15. The vasectomy reversal stent of claim 11, wherein the tube comprises an open end adjacent the first portion, the open end having an interior diameter that is greater than an interior diameter of the first portion.

16. (canceled)

17. The vasectomy reversal stent of claim 15, wherein the tube comprises a second open end opposite the open end, the second open end is adjacent to the second portion and has an exterior diameter that is less than the exterior diameter of the second portion.

18. (canceled)

19. The vasectomy reversal stent of claim 11, comprising a transition region disposed between the first portion and second portion, wherein an exterior diameter of the transition region gradually decreases from the exterior diameter of the first portion to the exterior diameter of the second portion.

20. The vasectomy reversal stent of claim 11, wherein a length of the first portion is less than a length of the second portion.

21-23. (canceled)

24. The vasectomy reversal stent of claim 19, wherein the loop is coupled to the exterior surface of the tube in the transition region.

25-26. (canceled)

27. The vasectomy reversal stent of claim 11, comprising a visual indicator disposed on the exterior surface of the tube at a position that is longitudinally offset from an open end of the tube.

28-39. (canceled)
